(19) United States
(12) Patent Application Publication
(10) Pub. No.: US 2022/0192651 A1
(43) Pub. Date: Jun. 23, 2022

(54) SINGLE PORT FOR MINIMALLY INVASIVE SURGERY

(71) Applicant: ORANGE MEDICS, INC., Daejeon (KR)

(72) Inventor: Ki Hwan LEE, Daejeon (KR)

(73) Assignee: ORANGE MEDICS, INC., Daejeon (KR)

(21) Appl. No.: 17/600,098

(22) PCT Filed: Oct. 8, 2020
(86) PCT No.: PCT/KR2020/013717

§ 371 Date: Sep. 29, 2021

(30) Foreign Application Priority Data
Feb. 5, 2020 (KR) 10-2020-0013662

(51) Int. Cl. A61B 17/02; A61B 17/34

(52) U.S. Cl. CPC A61B 17/0293; A61B 17/0218; A61B 17/3423; A61B 2017/3466; A61B 17/3462; A61B 2017/0287; A61B 2017/3445; A61B 17/3431

(57) ABSTRACT

A single port for minimally invasive surgery includes: a base having a cover guide, a first annular flange, and a knob; a channel unit having a second annular flange, a cover, and a plurality of introduction channels; a coupling unit securing the second annular flange to the first annular flange; a wound retractor having a wound retractor membrane, an outer ring, and an inner ring; and a connector connecting the wound retractor to the base.

### TECHNICAL FIELD

The present invention relates to single port for minimally invasive surgery, and more particularly to a single port for minimally invasive surgery, which provides enhanced usability during laparoscopic surgery.

### BACKGROUND ART

In general, laparoscopic surgery refers to a surgical procedure in which a small incision (hole), instead of a large opening as in traditional open surgery, is made in the abdomen and a gas for distension, a video camera, and various surgical instruments are inserted into the abdomen through the incision. Due to a much smaller incision size than traditional open surgery, laparoscopic surgery can ensure better cosmetic results of surgical wound closure and less incision-induced pain. In addition, due to quicker recovery than traditional open surgery, laparoscopic surgery has advantages of shorter hospital stay and quicker return to everyday life.

In order to prevent damage to an incision site by surgical instruments and gas leakage through an incision, a separate port is disposed in the incision.

However, since a conventional port is configured to guide only one surgical instrument, it is necessary to make incisions in a patient's abdomen according to the number of necessary surgical instruments (a gas injector, an endoscope, forceps, scissors, and the like).

In order to solve this problem, a single port access device including one port having a plurality of sleeves is being developed. In particular, a conventional single port access device includes a rigid body contacting one incision made in the abdomen, a plurality of sleeves disposed on the body, a guide tube formed on a lower surface of the body and inserted into the incision to closely contact a side surface of the incision, a gas inlet formed through the body and adapted for a gas to be injected into the abdominal cavity therethrough, and a gas outlet adapted for a gas to be discharged from the abdominal cavity therethrough.

However, such a conventional single port for laparoscopic surgery has a problem in that a gas injection pipe or a gas discharge pipe interferes with the view of an endoscopic camera or collides with surgical instruments in a confined space.

As an example of prior documents, there is Korean Patent Registration No. 1042305 (published on Jun. 17, 2011).

### DISCLOSURE

### Technical Problem

Embodiments of the present invention have been conceived to solve such a problem in the art and provide a single port for minimally invasive surgery, which provides enhanced usability during laparoscopic surgery.

It will be understood that aspects of the present invention are not limited to the above one. The above and other aspects of the present invention will become apparent to those skilled in the art from the detailed description of the following embodiments in conjunction with the accompanying drawings.

### Technical Solution

In accordance with one aspect of the present invention, there is provided a single port for minimally invasive surgery, including: a base having a cover guide formed with a first through-hole through which a surgical instrument passes, a first annular flange formed along an edge of the cover guide, and a plurality of knobs formed at predetermined intervals on the first annular flange in a circumferential direction of the first annular flange, each of the knobs having a hook protruding from a lower surface thereof toward a center of the base; a channel unit having a second annular flange closely contacting an upper surface of the first annular flange, a cover connected to the second annular flange and covering the base, and a plurality of introduction channels protruding from the cover and allowing passage of the surgical instrument therethrough; a coupling unit disposed on an upper surface of the second annular flange and securing the second annular flange to the first annular flange; a wound retractor having a wound retractor membrane partially inserted into an incision in a patient's abdomen and guiding insertion of the surgical instrument into a patient's abdominal cavity, an outer ring disposed at an upper portion of the wound retractor membrane, and an inner ring disposed at a lower portion of the wound retractor membrane; and a connector disposed between the outer ring and the knob and connecting the wound retractor to the base.

The connector may have a connector ring, a rib flange circumferentially protruding from an inner peripheral surface of the connector ring and closely contacting a lower surface of the outer ring, and a locking groove circumferentially formed on an outer peripheral surface of the connector ring and coupled to the hook.

In one embodiment of the present invention, the hook may be elastically deformed to expand outward by being pushed by the outer peripheral surface of the connector ring upon moving the connector toward the base and then may be returned to an original shape thereof by elastic restoring force to be coupled to the locking groove.

In one embodiment of the present invention, the single port for minimally invasive surgery may further include: a gas discharge pipe having one end coupled to the inner ring to be positioned inside the patient's abdominal cavity and the other end positioned outside the patient's abdominal cavity to guide discharge of gases from the patient's abdominal cavity, wherein the gas discharge pipe may include a discharge tube and an extension bar diametrically extending from one end of the discharge tube.

In addition, the gas discharge pipe may further include a locking protrusion protruding from both ends of the extension bar toward the one end of the discharge tube.

The inner ring may include a coupling hole formed through the inner ring in a height direction of the inner ring and allowing the discharge tube to be inserted thereinto, a slit formed by partially cutting out the inner ring and allowing the discharge tube to be inserted into the coupling hole from outside of the inner ring therethrough, and a first insertion groove formed on a lower surface of the inner ring and allowing the extension bar to be inserted thereinto.

In addition, the inner ring may further include a second insertion groove formed at both ends of the first insertion groove and allowing the locking protrusion to be inserted thereinto.

In one embodiment of the present invention, the base may further have a coupler radially protruding from an outer peripheral surface of the first annular flange and coupled to an injection tube guiding feed gas to be injected into the patient's abdominal cavity and an inflow guide hole radially formed through the first annular flange to be connected to the coupler, the inflow guide hole guiding the feed gas introduced into the coupler to move to an inside of the first annular flange.

In one embodiment of the present invention, the base may further have an annular plate disposed along an outer peripheral surface of the first annular flange, wherein the annular plate may have a curved portion defining a bending space into which the knob is bendable to separate the hook from the locking groove.

In one embodiment of the present invention, the first annular flange may have a plurality of first coupling holes formed at predetermined intervals in a circumferential direction of the first annular flange, the second annular flange may have a plurality of second coupling holes corresponding to the first coupling holes, and the coupling unit may have a plurality of coupling protrusions coupled to the first coupling holes through the second coupling holes, respectively.

In one embodiment of the present invention, the first annular flange may further have an alignment groove formed on the upper surface thereof and the second annular flange may further have an alignment protrusion formed at a lower surface thereof, such that the first coupling holes can be aligned with the second coupling holes, respectively, when the alignment groove is coupled to the alignment protrusion.

In one embodiment of the present invention, the inner ring may have a guide groove formed on an outer peripheral surface thereof and allowing the inner ring to be deformed into a straight line shape.

In one embodiment of the present invention, the guide groove may include a plurality of guide grooves symmetric to one another with respect to a center of the inner ring.

In one embodiment of the present invention, the single port for minimally invasive surgery may further include: a port unit disposed on an upper surface of each of the introduction channels and allowing passage of the surgical instrument therethrough, the port unit having a socket coupled to an upper end of the introduction channel and having a first port hole through which the surgical instrument passes, a valve coupled to the socket and having a pair of elastic gates inserted into the first port hole, the pair of elastic gates being adapted to open or close the first port hole, a core coupled to the socket from above the valve to secure the valve and having a second port hole through which the surgical instrument passes, a sealing cover disposed on an upper surface of the core and having a third port hole through which the surgical instrument passes, and a cap coupled to the socket, receiving the sealing cover and the core therein, and having a fourth port hole through which the surgical instrument passes.

In one embodiment of the present invention, the socket may further have a plurality of locking frames formed in a circumferential direction thereof and each having a locking hole, a seating hole formed between each pair of adjacent locking frames, and a first annular groove circumferentially formed under the locking frames, and the core may further have a first fitting protrusion coupled to the locking hole and a seating protrusion coupled to the seating hole.

### Advantageous Effects

According to embodiments of the present invention, the coupler radially protrudes from a side surface of the base. Accordingly, the injection tube connected to the coupler can extend laterally of the single port for minimally invasive surgery. In this way, it is possible to reduce collision between the injection tube and surgical instruments and to prevent the injection tube from being compressed or twisted during surgery.

In addition, according to embodiments of the present invention, the discharge tube can be easily coupled and secured to the inner ring by inserting the discharge tube into the coupling hole through the slit of the inner ring and inserting the extension bar and the locking protrusion into the first insertion groove and the second insertion groove, respectively. In addition, even when the gas discharge pipe is pulled upward, the discharge tube can remain secured to the inner ring without being separated from the inner ring.

Further, according to embodiments of the present invention, the inner ring has a guide groove formed on the outer peripheral surface thereof. The guide groove allows the inner ring to be deformed into a shape close to a straight line when the inner ring is compressed by external force, thereby facilitating insertion of the inner ring into an incision in a patient's abdomen during laparoscopic surgery.

It will be understood that advantageous effects of the present invention are not limited to the above ones, and include any advantageous effects conceivable from the features disclosed in the detailed description of the present invention or the appended claims.

### DESCRIPTION OF DRAWINGS

FIG. 1 is a perspective view of a single port for minimally invasive surgery according to one embodiment of the present invention.

FIG. 2 is a sectional view of the single port for minimally invasive surgery of FIG. 1, focused on a base of the single port.

FIG. 3 is a perspective view of the base of the single port for minimally invasive surgery of FIG. 1.

FIG. 4 is a perspective view of a channel unit of the single port for minimally invasive surgery of FIG. 1.

FIG. 5 is a perspective view of a coupling unit of the single port for minimally invasive surgery of FIG. 1.

FIG. 6 is a perspective view of a wound retractor of the single port for minimally invasive surgery of FIG. 1.

FIG. 7 is a perspective view of a connector of the single port for minimally invasive surgery of FIG. 1.

FIG. 8 is a perspective view of an inner ring of FIG. 7.

FIG. 9 is an assembly view of the inner ring of FIG. 8 and a gas discharge pipe.

FIG. 10 is a sectional view of a port unit of the single port for minimally invasive surgery of FIG. 1.

FIG. 11 is an exploded perspective view of the port unit of FIG. 10.

### BEST MODE

Reference will now be made in detail to various embodiments, examples of which are illustrated in the accompanying drawings. It should be understood that the present invention may be embodied in different ways and is not limited to the following embodiments. In the drawings, portions irrelevant to the description will be omitted for clarity. Like components will be denoted by like reference numerals throughout the specification.

Throughout the specification, when an element or layer is referred to as being “on,” “connected to,” or “coupled to” another element or layer, it may be directly on, connected to, or coupled to the other element or layer or intervening elements or layers may be present. In addition, unless stated otherwise, the term “includes” should be interpreted as not excluding the presence of other components than those listed herein.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof.

Hereinafter, embodiments of the present invention will be described with reference to the accompanying drawings.

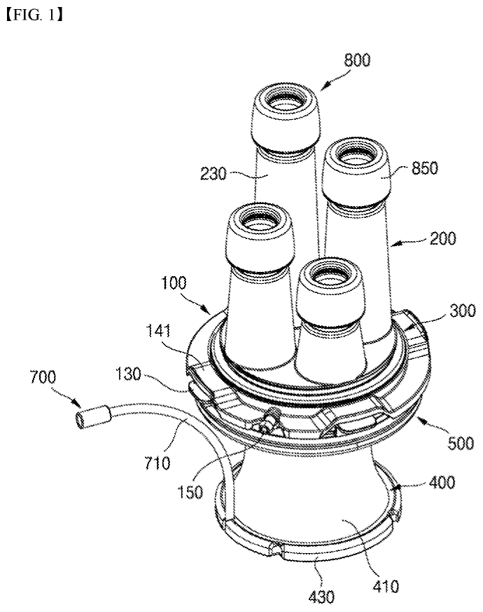

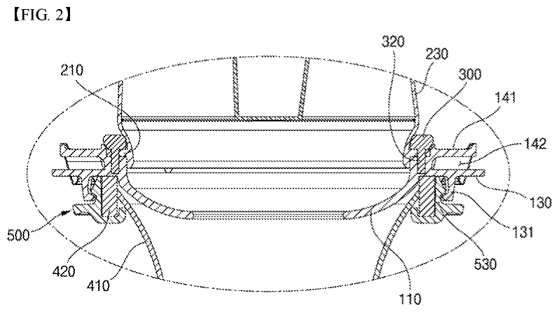

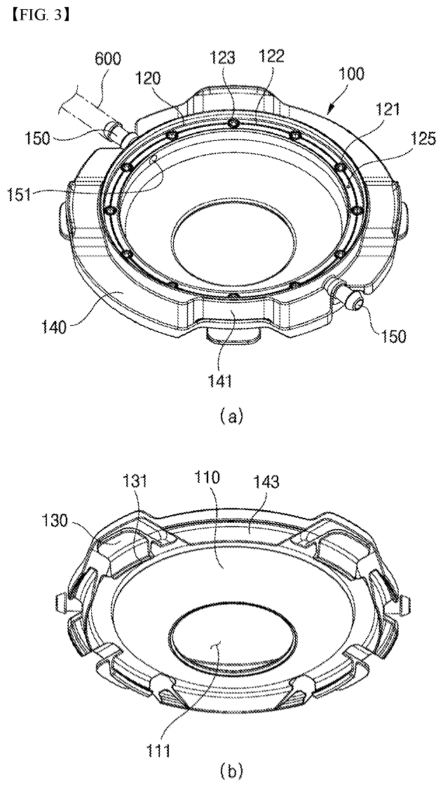

FIG. 1 is a perspective view of a single port for minimally invasive surgery according to one embodiment of the present invention, FIG. 2 is a sectional view of the single port for minimally invasive surgery of FIG. 1, focused on a base of the single port, and FIG. 3 is a perspective view of the base of the single port for minimally invasive surgery of FIG. 1.

Referring to FIG. 1 to FIG. 3, the single port for minimally invasive surgery includes a base 100, a channel unit 200, a coupling unit 300, a wound retractor 400, and a connector 500.

The base 100 may have a cover guide 110, a first annular flange 120, and a knob 130.

The cover guide 110 may have a first through-hole 111 through which a surgical instrument passes. Here, the surgical instrument may refer to all instruments used for surgery, such as an endoscope, forceps, and scissors. The cover guide 110 may have a concave shape to easily guide the surgical instrument to the first through-hole 111.

The first annular flange 120 may be formed along an edge of the cover guide 110. The first annular flange 120 may have a plurality of first coupling holes 121 formed on an upper surface thereof, wherein the first coupling holes 121 may be formed at predetermined intervals in a circumferential direction of the first annular flange 120.

In addition, the first annular flange 120 may have a first rib 122 and a second rib 123. The first rib 122 may protrude from the upper surface of the first annular flange 120 along the circumference of the first annular flange 120. The second rib 123 may be formed on the upper surface of the first annular flange 120 along an edge of the first through-hole 111.

The knob 130 may include a plurality of knobs 130 formed at predetermined intervals on the first annular flange 120 along the circumference of the first annular flange 120. The knob 130 may have a hook 131 protruding from a lower surface thereof toward the center of the first annular flange 120. The knob 130 may be resiliently bendable.

In addition, the base 100 may have a coupler 150 and an inflow guide hole 151. The coupler 150 may radially protrude from an outer peripheral surface of the first annular flange 120. The coupler 150 may be coupled to an injection tube 600 guiding feed gas to be injected into a patient's abdominal cavity. The inflow guide hole 151 may be radially formed through the first annular flange 120 and may be connected to the coupler 150. The feed gas delivered to the injection tube 600 and the coupler 150 may be moved to an inside of the first annular flange 120 through the inflow guide hole 151 and then may be moved through the first through-hole 111. Since the coupler 150 radially protrudes from the outer peripheral surface of the first annular flange 120, the injection tube 600 can extend laterally of the single port for minimally invasive surgery. In this way, it is possible to reduce collision between the injection tube 600 and a surgical instrument and to prevent the injection tube 600 from being compressed or twisted during surgery. In addition, the coupler 150 may include a plurality of coupling holes 150, whereby supply of the feed gas can be easily increased as necessary.

In addition, the base 100 may have an annular plate 140. The annular plate 140 may be disposed along the outer peripheral surface of the first annular flange 120. The annular plate 140 may have a curved portion 141 formed at a location corresponding to the knob 130. The curved portion 141 may be curved upward such that a bending space 142 is defined between the knob 130 and the curved portion 141. The bending space 142 may be a space allowing the knob 130 to be bendable upward. In addition, the annular plate 140 may have a plurality of skirts 143 protruding at predetermined intervals from a lower surface thereof in a circumferential direction thereof.

Preferably, the base 100 is formed of a material that is not excessively flexible while allowing elastic deformation of the knob 130, for example, polycarbonate (PC).

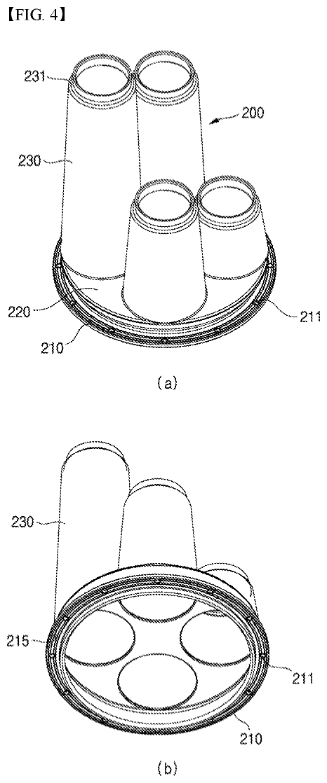

FIG. 4 is a perspective view of the channel unit of the single port for minimally invasive surgery of FIG. 1.

Referring further to FIG. 4, the channel unit 200 may have a second annular flange 210, a cover 220, and an introduction channel 230.

The second annular flange 210 may correspond in diameter and width to the first annular flange 120. In addition, the second annular flange 210 may have a plurality of second coupling holes 211. The second coupling holes 211 may correspond to the first coupling holes 121, respectively, and may be formed through the second annular flange 210.

The first annular flange 120 may further have an alignment groove 125 formed on an upper surface thereof, and the second annular flange 210 may have an alignment protrusion 215 formed on a lower surface thereof and coupled to the alignment groove 125. When the alignment protrusion 215 is coupled to the alignment groove 125 with the second annular flange 210 closely contacting the upper surface of the first annular flange 120, the first coupling holes 121 may be aligned with the second coupling holes 211, respectively.

The cover 220 may be connected to the second annular flange 210. With the second annular flange 210 closely contacting the first annular flange 120, the cover 220 may cover the base 100.

The introduction channel 230 may protrude from the cover 220 and may include a plurality of introduction channels. In addition, each of the introduction channels 230 may have a different height. The introduction channel 230 allows passage of a surgical instrument therethrough. Since each introduction channel 230 has a different height, mutual interference between surgical instruments can be reduced. The introduction channel 230 may have a stepped portion 231 formed at an upper end thereof.

The channel unit 200 may be formed of a material having a certain degree of flexibility, such as silicone or urethane.

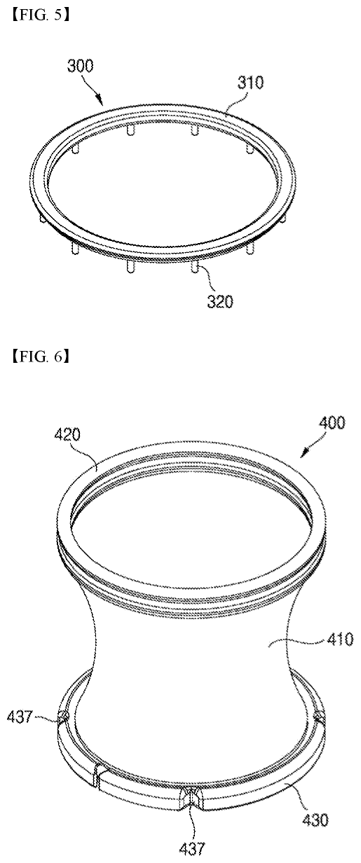

FIG. 5 is a perspective view of the coupling unit of the single port for minimally invasive surgery of FIG. 1.

Referring further to FIG. 5, the coupling unit 300 may have a pressure ring 310 corresponding in shape to the second annular flange 210 and a plurality of coupling protrusions 320 formed on a lower surface of the pressure ring 310 in a circumferential direction of the pressure ring. A space between each pair of adjacent coupling protrusions 320 may correspond to a space between each pair of adjacent second coupling holes 211. In addition, the coupling protrusions 320 may be fitted into the first coupling holes 121, respectively. The coupling unit 300 may be disposed on the upper surface of the second annular flange 210. When the coupling protrusions 320 are secured to the first coupling holes 121 through the second coupling holes 211 with the second annular flange 210 closely contacting the upper surface of the first annular flange 120, the channel unit 200 can be firmly coupled to the base 100. With the first annular flange 120 closely contacting the second annular flange 210, the first rib 122 and the second rib 123 of the first annular flange 120 closely contact the lower surface of the second annular flange 210, thereby enhancing sealing between the first annular flange 120 and the second annular flange 210. Preferably, the coupling unit 300 is formed of a material having a certain degree of strength, for example, poly carbonate (PC).

FIG. 6 is a perspective view of the wound retractor of the single port for minimally invasive surgery of FIG. 1.

Referring to FIG. 6, the wound retractor 400 may have a wound retractor membrane 410, an outer ring 420, and an inner ring 430.

The wound retractor membrane 410 may be a cylindrical elastic membrane. The wound retractor membrane 410 may be partially inserted into an incision in a patient's abdomen to guide insertion of a surgical instrument into a patient's abdominal cavity.

The outer ring 420 may be disposed at an upper portion of the wound retractor membrane 410 to be positioned outside the patient's abdominal cavity. The inner ring 430 may be disposed at a lower portion of the wound retractor membrane 410 to be inserted into the patient's abdominal cavity. The wound retractor 400 may be formed of urethane.

The connector 500 may be disposed between the outer ring 420 and the knob 130 to connect the wound retractor 400 to the base 100.

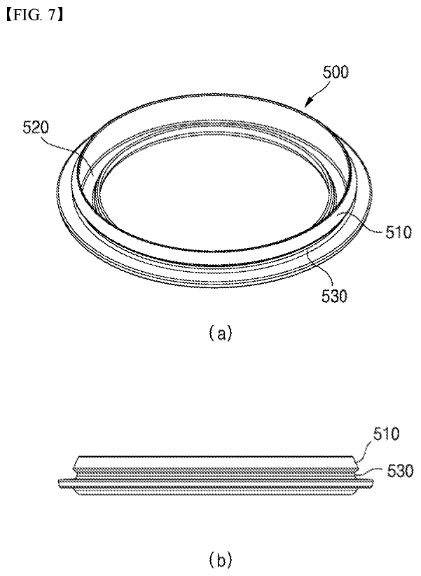

FIG. 7 is a perspective view of the connector of the single port for minimally invasive surgery of FIG. 1.

Referring further to FIG. 7, the connector 500 may have a connector ring 510, a rib flange 520, and a locking groove 530.

The rib flange 520 may protrude from an inner peripheral surface of the connector ring 510 in a circumferential direction of the connector ring 510. The outer ring 420 may be disposed inside the connector ring 510, and the rib flange 520 may closely contact the lower surface of the outer ring 420.

The locking groove 530 may be formed on an outer peripheral surface of the connector ring 510 in the circumferential direction of the connector ring 510. When the connector 500 is moved toward a lower portion of the base 100, that is, toward the first annular flange 120, with the outer ring 420 positioned inside the connector ring 510, the hook 131 is elastically deformed to expand outward by being pushed by the outer peripheral surface of the connector ring 510. The outer peripheral surface of the connector ring 510 may be increased in diameter toward a lower end thereof. In this way, the hook 131 can naturally expand outward upon moving the connector 500 upwards. Then, when the hook 131 reaches the locking groove 530, the hook 131 shrinks to an original shape thereof by elastic restoring force to be coupled to the locking groove 530. When the hook 131 is coupled to the locking groove 530, the upper surface of the outer ring 420 closely contacts the lower surface of the first annular flange 120 of the base 100. Here, the outer ring 420 closely contacts the rib flange 520, the connector ring 510, and the first annular flange 120, thereby preventing gas leakage. According to the present invention, the hook 131 can be caught in the locking groove 530 simply by a user pushing the connector 500 located under the base 100 toward the base 100, thereby facilitating coupling between the wound retractor 400 and the base 100.

When a user pulls the knob 130 upward with the hook 131 coupled to the locking groove 530, the knob 130 is bent into the bending space 142 and the hook 131 is released from the locking groove 530, causing the wound retractor 400 to be separated from the base 100. The curved portion 141 is formed above the knob 130 to allow a user to hold the curved portion 141 and the knob 130 together, thereby helping the user to easily bend the knob 130 upwards.

With the hook 131 coupled to the locking groove 530, the skirt 143 of the base 100 closely contacts the outer peripheral surface of the connector ring 510 to help the base 100 to be concentric with the connector 500. The connector 500 may be formed of poly carbonate (PC).

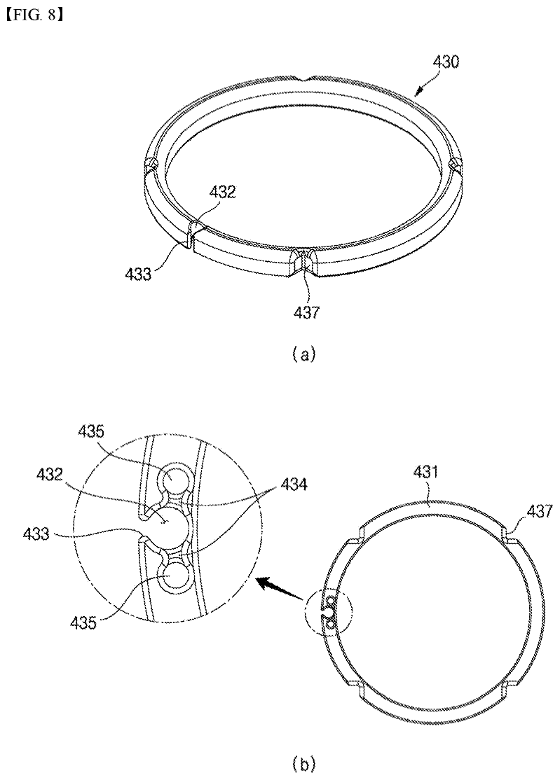

FIG. 8 is a perspective view of the inner ring of FIG. 7, and FIG. 9 is an assembly view of the inner ring of FIG. 8 and a gas discharge pipe.

Referring to FIG. 8 and FIG. 9, the single port for minimally invasive surgery may further include a gas discharge pipe 700.

The gas discharge pipe 700 may have a lower end coupled to the inner ring 430 to be positioned inside a patient's abdominal cavity. In addition, the gas discharge pipe 700 may have an upper end positioned outside the patient's abdominal cavity. The gas discharge pipe 700 serves to guide discharge of gases from the patient's abdominal cavity.

The gas discharge pipe 700 may have a discharge tube 710, an extension bar 720, and a locking protrusion 730.

The discharge tube 710 may have a length sufficient to guide discharge of gases from the patient's abdominal cavity.

The extension bar 720 may diametrically extend from a lower end of the discharge tube 710. The locking protrusion 730 may be formed at both ends of the extension bar 720 and may protrude toward an upper end of the discharge tube 710.

The inner ring 430 may have a coupling hole 432, a slit 433, a first insertion groove 434, and a second insertion groove 435.

The coupling hole 432 may be formed through the inner ring 430 in a height direction of the inner ring 430.

The slit 433 may be formed by partially cutting out the inner ring 430, that is, an inner ring body 431, and may be connected to the coupling hole 432. The coupling hole 432 may be connected to an outside of the inner ring 430 through the slit 433, such that the discharge tube 710 can be inserted into the coupling hole 432 from the outside of the inner ring 430.

The first insertion groove 434 may be formed on a lower surface 431 of the inner ring 430 to allow the extension bar 720 to be inserted thereinto.

The second insertion groove 435 may be formed at both ends of the first insertion groove 434 to allow the locking protrusion 730 to be inserted thereinto.

The gas discharge pipe 700 can be easily coupled and secured to the inner ring 430 without being moved upwardly of the inner ring 430 by inserting the discharge tube 710 into the coupling hole 432 through the slit 433 and inserting the extension bar 720 and the locking protrusion 730 into the first insertion groove 434 and the second insertion groove 435, respectively. That is, even when the gas discharge pipe 700 is pulled upward, the gas discharge pipe 700 can remain coupled to the inner ring 430 without being separated from the inner ring 430.

In addition, the inner ring 430 may have a guide groove 437. The guide groove 437 may be formed on the outer peripheral surface of the inner ring 430. In cross-sectional view perpendicular to the height direction of the inner ring 430, the inner ring 430 may have a reduced width at a portion formed with the guide groove 437. The guide groove 437 allows the inner ring 430 to be deformed into a straight line shape, more specifically, into a shape close to a straight line, when the inner ring 430 is compressed by external force. In laparoscopic surgery, a user compresses the inner ring 430 before inserting the inner ring 430 into an incision in a patient's abdomen. When the inner ring 430 is deformed into a shape close to a straight line by means of the guide groove 437, the inner ring 430 can be more easily inserted into the incision. The guide groove 437 may include a plurality of guide grooves, wherein the guide grooves 437 may be symmetric to one another with respect to the center of the inner ring 430. As the number of guide grooves 437 increases, the inner ring 430 can be deformed into a shape closer to a straight line.

Referring to FIG. 1 and FIG. 2, the single port for minimally invasive surgery may further include a port unit 800. The port unit 800 may be disposed on the upper surface of the introduction channel 230 and may allow passage of a surgical instrument therethrough.

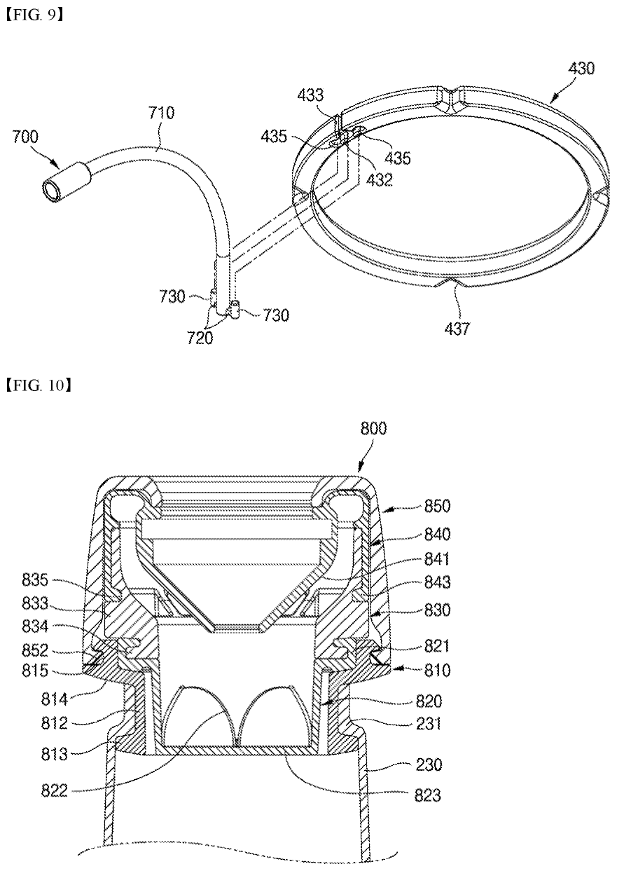

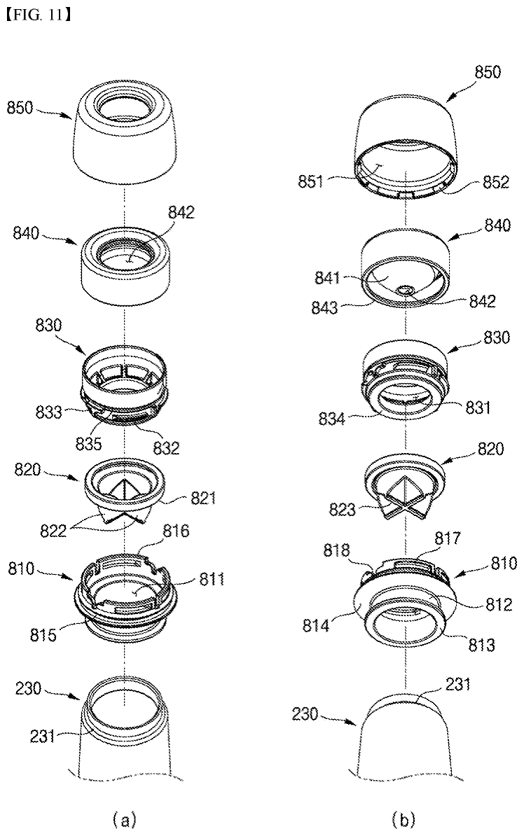

FIG. 10 is a sectional view of the port unit of the single port for minimally invasive surgery of FIG. 1, and FIG. 11 is an exploded perspective view of the port unit of FIG. 10.

Referring further to FIG. 10 and FIG. 11, the port unit 800 may have a socket 810, a valve 820, a core 830, a sealing cover 840, and a cap 850.

The socket 810 may be coupled to an upper end of the introduction channel 230 and may have a first port hole 811 through which a surgical instrument passes. Specifically, the socket 810 may have an annular body 812, a first locking portion 813 formed on a lower surface of the annular body 812, and a second locking portion 814 formed on an upper surface of the annular body 812. The annular body 812 of the socket 810 may be inserted into the upper end of the introduction channel 230, and the first locking portion 813 may be caught by the stepped portion 231 of the introduction channel 230 to prevent the socket 810 from being separated from the introduction channel 230. The second locking portion 814 may closely contact the upper end of the introduction channel 230, whereby the socket 810 can be stably coupled to the upper end of the introduction channel 230.

In addition, the socket 810 may have a first annular groove 815 and a locking frame 816. The first annular groove 815 may be formed on an upper surface of the second locking portion 814 in a circumferential direction of the second locking portion. The locking frame 816 may include a plurality of locking frames 816 formed in a circumferential direction of the socket 810 and each having a locking hole 817 formed therethrough. In addition, the socket 810 may have a seating hole 818 formed between a pair of adjacent locking frames 816.

The valve 820 may be coupled to an inner surface of the socket 810 and may have a third locking portion 821 and a pair of elastic gates 822.

The third locking portion 821 may be formed at an upper surface of the valve 820 and may be seated on an inner surface of the second locking portion 814 of the socket 810.

The pair of elastic gates 822 may be symmetric to each other with respect to the center of the valve 820. Each of the pair of elastic gates 822 may be concave toward a lower end thereof, and lower ends of the pair of elastic gates 822 may closely contact each other.

The valve 820 may be formed of silicone. When the pair of elastic gates 822 is in an initial state thereof, the lower ends of the pair of elastic gates 822 may closely contact each other. When the pair of elastic gates 822 is subjected to external force, the pair of elastic gates 822 may be deformed such that the lower ends of the pair of elastic gates 822 are separated from each other. In this way, the pair of elastic gates 822 can be switched between an open position and a closed position.

In addition, the valve 820 may have a rigid rib 823 formed along the lower ends of the pair of elastic gates 822. The rigid rib 823 may be integrally formed with the pair of elastic gates 822. The rigid rib 823 provides restoring force allowing the lower ends of the pair of elastic gates 822 to closely contact each other and reduces the risk of damage to the lower ends of the pair of elastic gates 822 upon moving the pair of elastic gates 822 to the open position.

The core 830 may be coupled to the socket 810 from above the valve 820 to secure the valve 820, and may have a second port hole 831 through which a surgical instrument passes. Specifically, the core 830 may have a fourth locking portion 834 protruding from a lower end thereof. The fourth locking portion 834 may be coupled to an inner surface of the third locking portion 821 of the valve 820 to secure the valve 820. In addition, the core 830 may have a plurality of first fitting protrusions 832 formed in a circumferential direction thereof with a space therebetween and a seating protrusion 833 formed between each pair of adjacent first fitting protrusions 832. The first fitting protrusions 832 may correspond to the locking holes 817 of the socket 810, respectively, and the seating protrusion 833 may correspond to the seating hole 818 of the socket 810. That is, the first fitting protrusions 832 may be coupled to the locking holes 817 and the seating protrusion 833 may be coupled to the seating hole 818, whereby the core 830 can be firmly coupled to the socket 810. The core 830 may have a second annular groove 835 circumferentially formed on an outer peripheral surface thereof.

The sealing cover 840 may be disposed on an upper surface of the core 830 and may have a sealing membrane 841. The sealing membrane 841 may be concave downwards and may be formed at a center thereof with a third port hole 842 through which a surgical instrument passes. The sealing cover 840 may have a fifth locking portion 843 formed in a circumferential direction thereof and coupled to the second annular groove 835 of the socket 810.

The cap 850 may be coupled to the socket 810 and may receive the sealing cover 840 and the core 830 therein. The cap 850 may have a fourth port hole 851 through which a surgical instrument passes. The cap 850 may have a second fitting protrusion 852 formed on an inner surface of a lower end thereof. The second fitting protrusion 852 may be coupled to the first annular groove 815 of the socket 810, whereby the cap 850 can be firmly coupled to the socket 810.

A surgical instrument introduced into the fourth port hole 851 of the cap 850 may be inserted into the introduction channel 230 through the third port hole 842 of the sealing cover 840, the second port hole 831 of the core 830, the pair of elastic gates 822 of the valve 820, and the first port hole 811 of the socket 810. The pair of elastic gates 822 may closely contact an outer peripheral surface of the surgical instrument by elastic restoring force, thereby preventing gas leakage.

Although some embodiments have been described herein, it should be understood that these embodiments are provided for illustration only and are not to be construed in any way as limiting the present invention, and that various modifications, changes, alterations, and equivalent embodiments can be made by those skilled in the art without departing from the spirit and scope of the invention. For example, components described as implemented separately may also be implemented in combined form, and vice versa.

The scope of the present invention is indicated by the following claims and all changes or modifications derived from the meaning and scope of the claims and equivalents thereto should be construed as being within the scope of the present invention.

### INDUSTRIAL APPLICABILITY

Laparoscopic surgery uses a single port access device including one port having a plurality of sleeves to ensure minimally invasive surgery. In particular, the single port for minimally invasive surgery according to the present invention provides enhanced ease of use and can be widely used in robotic surgery as well as simple laparoscopic surgery.

1. A single port for minimally invasive surgery, comprising:
a base having a cover guide formed with a first through-hole through which a surgical instrument passes, a first annular flange formed along an edge of the cover guide, and a plurality of knobs formed at predetermined intervals on the first annular flange in a circumferential direction of the first annular flange, each of the knobs having a hook protruding from a lower surface thereof toward a center of the base;
a channel unit having a second annular flange closely contacting an upper surface of the first annular flange, a cover connected to the second annular flange and covering the base, and a plurality of introduction channels protruding from the cover and allowing passage of the surgical instrument therethrough;
a coupling unit disposed on an upper surface of the second annular flange and securing the second annular flange to the first annular flange;
a wound retractor having a wound retractor membrane partially inserted into an incision in a patient's abdomen and guiding insertion of the surgical instrument into a patient's abdominal cavity, an outer ring disposed at an upper portion of the wound retractor membrane, and an inner ring disposed at a lower portion of the wound retractor membrane; and
a connector disposed between the outer ring and the knob and connecting the wound retractor to the base.

2. The single port for minimally invasive surgery according to claim 1, wherein the connector has a connector ring, a rib flange circumferentially protruding from an inner peripheral surface of the connector ring and closely contacting a lower surface of the outer ring, and a locking groove circumferentially formed on an outer peripheral surface of the connector ring and coupled to the hook.

3. The single port for minimally invasive surgery according to claim 2, wherein the hook is elastically deformed to expand outward by being pushed by the outer peripheral surface of the connector ring upon moving the connector toward the base and then is returned to an original shape thereof by elastic restoring force to be coupled to the locking groove.

4. The single port for minimally invasive surgery according to claim 1, further comprising:
a gas discharge pipe having one end coupled to the inner ring to be positioned inside the patient's abdominal cavity and the other end positioned outside the patient's abdominal cavity to guide discharge of gases from the patient's abdominal cavity,
wherein the gas discharge pipe comprises a discharge tube and an extension bar diametrically extending from one end of the discharge tube.

5. The single port for minimally invasive surgery according to claim 4, wherein the inner ring comprises
a coupling hole formed through the inner ring in a height direction of the inner ring and allowing the discharge tube to be inserted thereinto,
a slit formed by partially cutting out the inner ring and allowing the discharge tube to be inserted into the coupling hole from outside of the inner ring therethrough, and
a first insertion groove formed on a lower surface of the inner ring and allowing the extension bar to be inserted thereinto.

6. The single port for minimally invasive surgery according to claim 5, wherein the gas discharge pipe further comprises a locking protrusion protruding from both ends of the extension bar toward the one end of the discharge tube, and the inner ring further comprises a second insertion groove formed at both ends of the first insertion groove and allowing the locking protrusion to be inserted thereinto.

7. The single port for minimally invasive surgery according to claim 1, wherein the base further has a coupler radially protruding from an outer peripheral surface of the first annular flange and coupled to an injection tube guiding feed gas to be injected into the patient's abdominal cavity and an inflow guide hole radially formed through the first annular flange to be connected to the coupler, the inflow guide hole guiding the feed gas introduced into the coupler to move to an inside of the first annular flange.

8. The single port for minimally invasive surgery according to claim 1, wherein the base further has an annular plate disposed along an outer peripheral surface of the first annular flange, the annular plate having a curved portion defining a bending space into which the knob is bendable to separate the hook from the locking groove.

9. The single port for minimally invasive surgery according to claim 1, wherein:
the first annular flange has a plurality of first coupling holes formed at predetermined intervals in a circumferential direction of the first annular flange;
the second annular flange has a plurality of second coupling holes corresponding to the first coupling holes; and
the coupling unit has a plurality of coupling protrusions coupled to the first coupling holes through the second coupling holes, respectively.

10. The single port for minimally invasive surgery according to claim 9, wherein the first annular flange further has an alignment groove formed on the upper surface thereof and the second annular flange further has an alignment protrusion formed at a lower surface thereof, such that the first coupling holes are aligned with the second coupling holes, respectively, when the alignment groove is coupled to the alignment protrusion.

11. The single port for minimally invasive surgery according to claim 1, wherein the inner ring has a guide groove formed on an outer peripheral surface thereof and allowing the inner ring to be deformed into a straight line shape.

12. The single port for minimally invasive surgery according to claim 11, wherein the guide groove comprises a plurality of guide grooves symmetric to one another with respect to a center of the inner ring.

13. The single port for minimally invasive surgery according to claim 1, further comprising:
a port unit disposed on an upper surface of each of the introduction channels and allowing passage of the surgical instrument therethrough, the port unit having a socket coupled to an upper end of the introduction channel and having a first port hole through which the surgical instrument passes, a valve coupled to the socket and having a pair of elastic gates inserted into the first port hole, the pair of elastic gates being adapted to open or close the first port hole, a core coupled to the socket from above the valve to secure the valve and having a second port hole through which the surgical instrument passes, a sealing cover disposed on an upper surface of the core and having a third port hole through which the surgical instrument passes, and a cap coupled to the socket, receiving the sealing cover and the core therein, and having a fourth port hole through which the surgical instrument passes.

14. The single port for minimally invasive surgery according to claim 13, wherein the socket further has a plurality of locking frames formed in a circumferential direction thereof and each having a locking hole, a seating hole formed between each pair of adjacent locking frames, and a first annular groove circumferentially formed under the locking frames, and the core further has a first fitting protrusion coupled to the locking hole and a seating protrusion coupled to the seating hole.
